(19) United States
(12) Patent Application Publication
(10) Pub. No.: US 2005/0010080 A1
(43) Pub. Date: Jan. 13, 2005

(54) Electrode for use in urological resectoscopes

(76) Inventor: Jorg Dickopp, Reinbeck (DE)

Correspondence Address: RANKIN, HILL, PORTER & CLARK, LLP, 925 EUCLID AVENUE, SUITE 700, CLEVELAND, OH, 44115-1405, US

(21) Appl. No.: 10/485,907

(22) PCT Filed: Jun. 17, 2002
(86) PCT No.: PCT/EP02/06647

(30) Foreign Application Priority Data
Aug. 10, 2001 (DE) 101 39 449.7

(51) Int. Cl. A61B 1/00

(52) U.S. Cl. 600/105

(57) ABSTRACT

An electrode (9,) for use in urological resectoscopes including an elongated electrode carrier (7, 18, 19) that is provided in its proximal region with a spring element (20, 30′, 39, 40, 45) in order to establish an electric contact (12) and to mechanically fasten a sliding body (5) in a receiving guide (6, 36) via a snap-in connection. The spring element (20, 30′, 39, 40, 45) is disposed on the electrode body (7, 18, 19).

### BACKGROUND OF THE INVENTION

Urological resectoscopes have, within an elongate tubular shaft, an elongate, tubular optical system and an elongate electrode carrier, disposed on the distal end of which in the field of view of the optical system there is an HF electrode, generally constructed in the form of a cutting blade. The electrode carrier projects, together with the optical system, proximally beyond the shaft region of the resectoscope and is connected there to a sliding body, which is slidably mounted e.g. on the optical tube and is actuated back and forth by the operator to move the electrode longitudinally.

Provided in the sliding body is a contacting device, with which a conductive wire extending longitudinally through the electrode carrier to the electrode is contacted. Also provided is a fastener with which the electrode carrier may be fastened to the sliding body.

In constructions of the type referred to above, the proximal end region of the rod-shaped electrode carrier is inserted into a receiving guide on the sliding body, which is generally constructed in the form of a receiving bore. Fastening is effected by means of a locking connection with a spring element.

In the prior art, the spring element is constructed, e.g. in the form of a blade mounted resiliently transversely to the electrode carrier which springs lockingly into a peripheral groove in the electrode carrier. A disadvantage is that in the event of a defect on the spring element, the sliding body must be replaced, which is extremely complex in a urological resectoscope.

The object of the present invention resides in improving the possibilities for repairing a defective spring element.

### SUMMARY OF THE INVENTION

The electrode in accordance with the invention has a spring element on the electrode carrier. A simple edge on the sliding body in the region of the fastener is sufficient, behind which the spring element can move out resiliently to lock it, when it is inserted. If defects occur on the spring element, the electrode is replaced but is in any event replaced after each operation. As a result of the constant use of new spring elements, a constantly optimal function is thus ensured.

The spring element on the electrode carrier can advantageously be constructed to move radially outwardly under its resilience, e.g. with resiliently supported locking balls or the like. One or more spring tongues are provided which can be fabricated substantially more simply in a necessary small size in the region of about 1 mm. If only one spring tongue or some other spring element is provided on the electrode carrier at a predetermined peripheral angular position and if the edge is situated only on one side adjacent the electrode carrier, the precisely matching angular position of the one spring element must be ensured. If, on the other hand, a plurality of tongues or spring elements are provided distributed over the periphery, one of these always comes into engagement so that the exact angular position is not critical. This facilitates the installation of the spring tongues on the electrode carrier, e.g. by an arrangement independent of peripheral angle.

The spring tongues can, for instance, be secured to a metallic end member of the electrode carrier by riveting or soldering. The spring tongues are constructed as portions of a sleeve, which slides onto the electrode carrier and is secured by clamping, which makes manufacture substantially easier.

An end stop is preferably formed on the electrode carrier, which, in the opposite direction to the locking connection preventing withdrawal, ensures a secure mounting of the electrode carrier in the sliding body in both sliding directions.

The peripherally thickened portion, which is provided adjacent to the outwardly projecting spring element, a sharp edged spring tongue, provides protection against injury on the sharp edge spring tongue, for instance when touching it with the finger or when moving through a seal which seals the electrode carrier. A locking element can engage in the space between the spring element and peripheral thickened portion which affords the edge towards the spring element and engages the peripheral thickened portion, constituting the end stop, with a proximally situated further edge.

The longitudinally elastic hose can push the ring constituting the peripheral thickened portion into engagement with the spring element, a plurality of spring tongues, so that sharp edged spring tongues are completely shielded without injury by the engaging ring. If the electrode carrier is inserted, the spring tongues move in resiliently at the edge and the locking element carrying the edge pushes the ring before it, whilst shortening the longitudinally elastic hose, until it is locked in between the ring and spring tongues.

Instead of a radially resilient spring element, a rotationally resilient spring element can advantageously be provided which carries a radial projection, which is movable rotationally resiliently into and out of engagement with a fixed projection. A portion of the electrode carrier itself can bring about the rotational resilience in a structurally very simple manner, with respect to the distal end region of the electrode carrier, which, in the conventional construction of recectoscopes, is rotationally fixedly mounted on its optical tube.

The edge on the receiving guide with an empty space situated proximally thereof constitutes a distal abutment for the spring element which has moved resiliently outwards. It can be of very simple and stable construction so that long term functional security is ensured.

The edge can be of oblique shape so that, when the electrode carrier is pulled strongly, the spring element is forced radially inwards and the electrode carrier can be withdrawn. The edge can be moved out of engagement with the spring element by moving the locking element. The edge can therefore be of perpendicular construction. The result of this is that with the edge in the engaged position unintentional release of the electrode carrier is precluded but with the edge moved out of engagement the electrode carrier is very easily removable.

At one of its longitudinal ends, the elongate hole in the slider affords the edge, which ensures the locking engagement with the spring element. The edge can be moved out of engagement by movement of the slider. The elongate hole can, for instance, be broadened in its other end region such that it permits the electrode carrier to pass through freely with all the spring tongues, even in the state in which they have moved resiliently outwardly.

When passing through the narrow elongate hole, the spring tongues move in and move resiliently out in the region of the broadened step, where they lock against reverse movement. If the slider is moved in the direction of the elongate hole, the portion of the elongate hole where no step is present moves into engagement with the spring tongues. The broadened side wall of the step narrows down to the normal width of the elongate hole so that when the elongate hole is moved the spring tongues engage the electrode carrier and the latter can then be withdrawn with the electrode carrier.

The invention is schematically illustrated by way of example in the drawings

### BRIEF DESCRIPTION OF THE FIGURES

FIG. 1 is a representation of the invention showing a resectoscope with a mounted electrode carrier in side view,

FIG. 2 is an enlarged sectional view on the line 2-2 in FIG. 1,

FIG. 3 is a sectional view on the line 3-3 in FIG. 2,

FIG. 4 is a detailed view of the slider shown in FIG. 2 in the region of the elongate hole,

FIG. 5 is a scrap view similar to FIG. 3 of another embodiment,

FIG. 6 is a view similar to FIG. 3 of a further embodiment,

FIG. 7a, 7b are views similar to FIG. 3 of a further embodiment in two positions,

FIG. 8a, 8b are views similar to FIG. 2 of another embodiment in two positions of the slider,

FIG. 9 is a sectional view similar to FIG. 2 of a further embodiment,

FIG. 10 is a side view of the embodiment in FIG. 9,

FIG. 11 is a perspective view of a further embodiment of the spring element,

FIG. 12 is a side view of yet another further embodiment of the spring element,

FIG. 13 is a side view of the locking engagement of a further modification with rotary springing and

FIG. 14 is a proximal axial view of the embodiment of FIG. 13.

### DETAILED DESCRIPTION OF PREFERRED AND ALTERNATE EMBODIMENTS

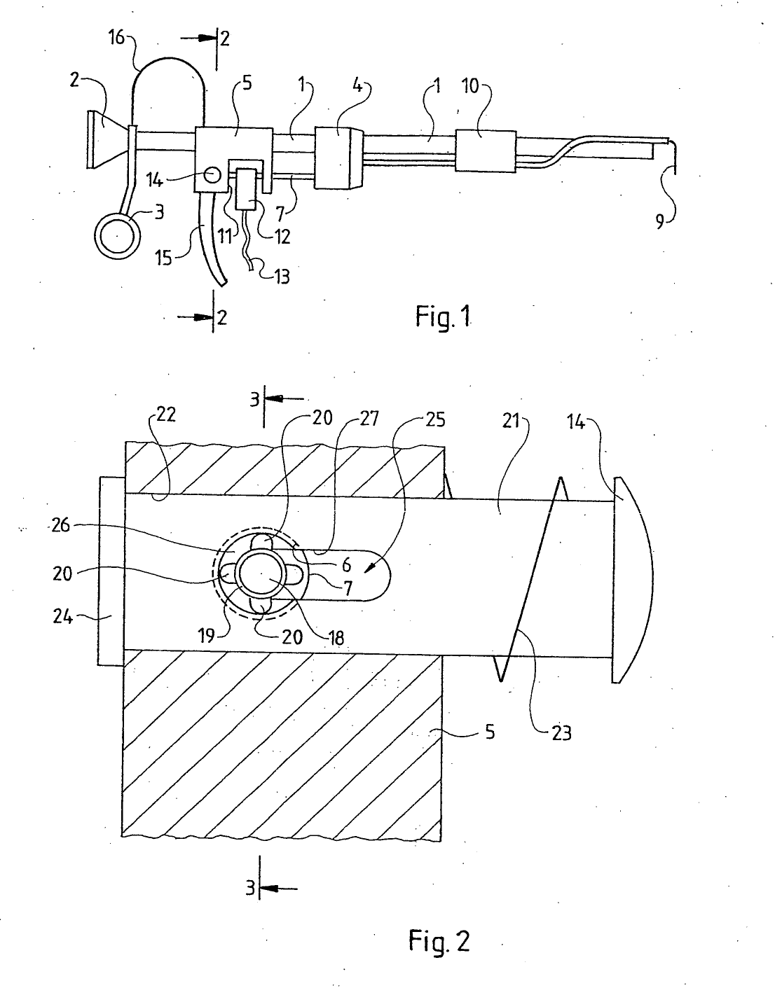

FIG. 1 shows, in conjunction with the associated detailed views in FIGS. 2-4, a substantially commercially standard resectoscope in side view. A continuous optical tube 1 carries an eyepiece 2 and a thumb ring 3 at the proximal end. Spaced therefrom, it carries a main body 4. A tubular shaft, which is not shown, may be connected to the latter, which surrounds the portion of the optical tube 1 positioned distally from the main body 1 at a spacing.

Slidably mounted on the optical tube 1 in the region between the thumb ring 3 and main body 4 is a sliding body 5. Inserted into the sliding body 5, in a receiving guide, which extends parallel to the optical tube 1 and is in the form of a receiving bore 6, is an electrode carrier 7, which passes from the sliding body 5 extending distally through a commonly gently bent passage in the main body 4, sealed by a seal 8, and extends from there distally in the direction parallel to the optical tube 1. At its distal end, the electrode carrier 7 carries an electrode 9 in the form of a conventional cutting blade and it is also mounted on the optical tube 1 with the conventional sliding sleeve 10 for the purpose of better guiding.

In the illustrated embodiment, a cutout 11 in the sliding body 5 exposes a portion of the electrode carrier 7 to the exterior. At this point, the illustrated clamping plug 12 can contact a contact member or, in the case of a bipolar electrode, two contact members, on the electrode carrier 7 and produce the electrical connection, via the illustrated cable 13, to one or two conductive wires, which are insulated within the electrode carrier 7 and extend to the electrode 9.

In order to replace the electrode 9, which is subjected to heavy wear, the electrode carrier 7 can be withdrawn, e.g. after removing the clamping plug 12 and after actuating a push button 14.

Tissue can be cut with the resectoscope seen in FIG. 1, when it is completed with the tubular shaft, which is not shown, by retracting movements of the electrode 9 whilst it is acted on by high frequency. In order to perform this retracting movement, the operator engages the thumb ring 3 with his thumb and a finger grip 15, which is connected to the sliding body 5, with his index finger and pulls it in the proximal direction against the force of a restoring leaf spring 16, which is fastened on the one hand to the thumb grip and on the other hand to the sliding body 5.

The electrode 9 is subjected to heavy wear. The contact points at which the clamping plug 12 engage also have a tendency to wear. The electrode carrier 7 with the electrode 9 is thus replaced after each operation. Replacement occurs after pressing in the push button 14. A new electrode carrier 7 is inserted from the distal end and secured in position with a self-locking fastener. This will be explained below with reference to FIGS. 2 to 4.

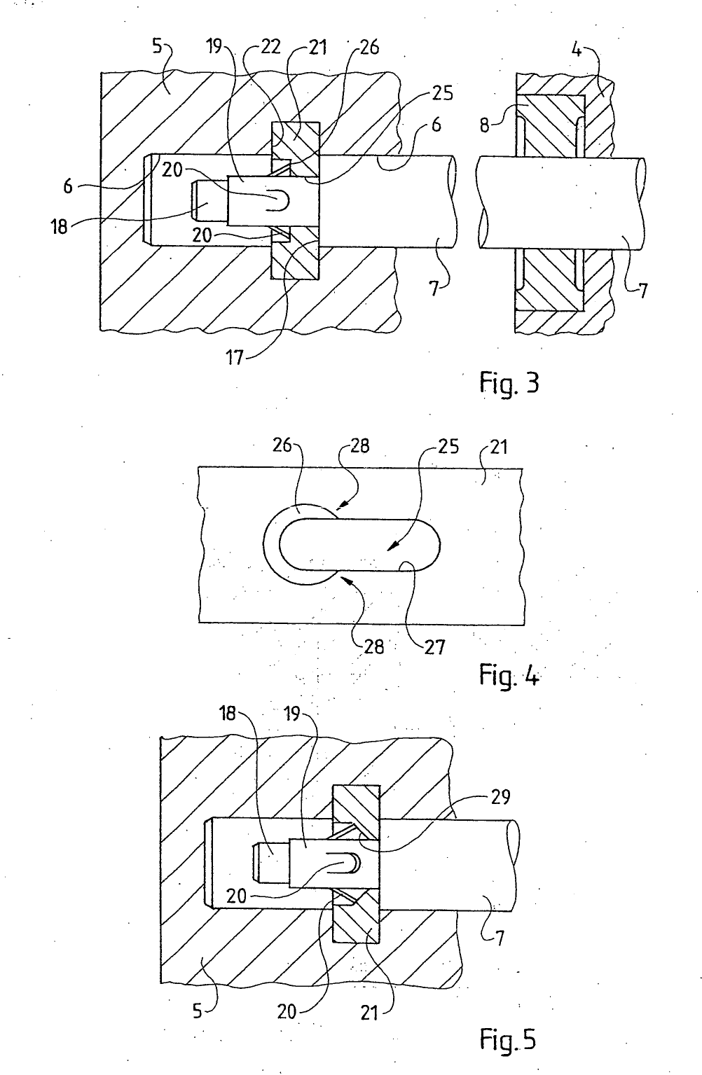

The proximal end region of the electrode carrier 7 is shown in FIG. 3. At an engagement step 17 of a peripheral thickened portion, its diameter reduces down to a thin end member 18, which preferably consists of metal. Clampingly slid onto the end member 18 until it engages the step 17 is a tubular sleeve 19 of spring metal. The sleeve 19 has four spring tongues 20, which are peripherally spaced by 90° and are each cut out by a half round cut in the sleeve 19 and are preformed obliquely outwardly so that their free ends are directed obliquely outwardly and distally. When loaded from the exterior, they can resiliently engage the surface of the end member 18.

The push button 14 is situated at the end of a slider 21 which, as shown in FIG. 3, is of rectangular flat cross-section. The slider 21 passes through the sliding body 5 in a rectangular passage 22 of corresponding cross-section. The push button 14 is supported against the sliding body 5 with a helical spring 23. At the other end of the slider 21, on the other side of the sliding body 5, the slider carries an end stop 24. It is secured in the locked position shown in FIG. 2 by the spring 23 and can be moved to the left as shown in FIG. 2, in the illustrated exemplary embodiment, by actuating the push button 14.

Formed in the slider 21 is an elongate hole 25, which passes through it in the direction of the receiving bore 6. As shown, in particular, in FIG. 4, the elongate hole 25 is provided, at the left-hand end in this figure, with a step 26 which increases the breadth of the elongate hole 25 and is formed as a circular depression centrally with respect to the radius of the left-hand end of the elongate hole. The remaining region 27 of the elongate hole 25 extends with constant breadth through the entire thickness of the slider 21.

When the electrode carrier 7 is inserted (from the right in FIG. 3) into the receiving bore 6, the slider 21 is not moved. The end member 18 of the electrode carrier 7 moves with the sleeve 19 through the elongate hole 25 in the region of the step 26. The spring tongues 20 move in resiliently so that they can pass through the narrow elongate hole 25. When the tongues 20 are pushed in, they move into the region of the broadened space at the step 26 and thus expand outwardly and engage the step 26. At the same time, the abutment step 17 comes into engagement with the distal surface of the slider 21. The electrode carrier 7 is thus self-lockingly secured to the sliding body 5 in a tension and pressure-resistant manner.

If the electrode carrier 7 is to be withdrawn again, the slider 21 is pushed in with the push button 14 until the other end of the elongate hole 25 in the region 27 surrounds the end member 18 of the electrode carrier 7. When this movement occurs, the tongues 20 at the top and bottom in FIG. 2 are positioned in the region of the oblique transition of the step 26 with the remaining region 27 of the elongate hole 25. This oblique region is marked in FIG. 4 with the arrows 28. The electrode carrier 7 can now be withdrawn.

Within the main body 4, the end member 18 with the tongues 20 must pass through the seal 8 shown in the right-hand portion of FIG. 3. Damage to the seal 8 by the tongues 20 is avoided by the fact that the seal 8, as shown in FIG. 3, is wider than the distance between the abutment edge 17 and the tongues 20. When the tongues are withdrawn through the seal 8, they thus do not come into damaging engagement.

FIG. 5 shows a modified embodiment which substantially corresponds to the left-hand portion of FIG. 3. The same reference numerals are thus used insofar as possible.

In this case, only the step 26 is of different shape. In the embodiment of FIGS. 2-4, this step is of right-angled shape, In the embodiment of FIG. 5, it extends taperingly in the distal direction with a funnel-shaped bevel 29.

When the electrode carrier 7 is retracted, the tongues 20 are forced inwards at the bevel 29 and can be moved in under the action of an increased force and pulled through. The slider 21 can thus be of immovably fixed construction in this embodiment. The step with the bevel 29 can also be formed directly in the sliding body 5, which should be made from suitably hard material. An elongate hole may also be dispensed within this construction. The bevel 29 can be of a circular shape symmetrical with respect to the axis of the electrode carrier 7 shown in FIG. 5.

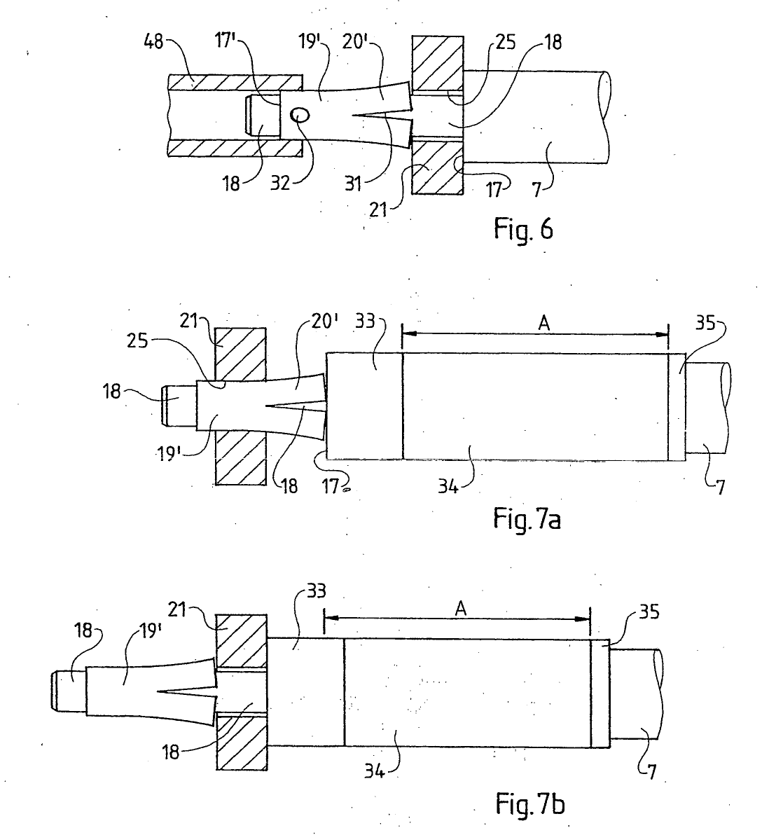

FIG. 6 is a view corresponding to FIG. 3 and shows a further embodiment of the invention. In this case, only the sleeve 19′ with the tongues 20′ are of different construction. Formed from the distal end of the sleeve 19′ and distributed over its periphery are slots 31, which are parallel to the axis and divide the distal end region of the sleeve 19′ into two or more tongues 20′, which are prefabricated to be bent gently outwards and can resiliently engage the end member 18 of the electrode carrier 7. The sleeve 19′ can be fastened to the end member 18 with a push-in member 32.

The sleeve 19 can, as also can the sleeve 19 in FIGS. 2 and 3, be constructed longer and closed at its proximal end.

FIGS. 7a and 7b show a further embodiment, which corresponds to the sleeve 19′ of the embodiment of FIG. 6. The abutment step 17 is, however, formed in this embodiment on a ring 33, which constitutes the peripheral thickened portion of the electrode carrier 7 protecting the sharp edged distal ends of the spring tongues 20.

The ring 33 is slidably mounted on the electrode carrier 7 and is supported on the proximal end of a longitudinally elastic hose 34, which surrounds the electrode carrier 7 and bears with its distal end on an external flange 35 fixed to the electrode carrier 7.

In FIG. 7a, the hose 34 has moved outwardly under its resilience in the longitudinal direction to its length A. It thus presses the ring 33 into engagement with the tongues 20′ on the sleeve 19′.

If, in the transition from the position shown in FIG. 7a to that shown in FIG. 7b, the electrode carrier 7 is slid in a proximal direction, that is to say to the left with respect to the slider 21 in the figures, the tongues 20′ move resiliently inwards and through the elongate hole 25 in the slider 21 until they spring outwardly locking behind the slider 21, as shown in FIG. 7b. The distal side of the slider 21 thus pushes the ring 33 in front of it with an elastic reduction in length of the hose 34 so that in the locked position shown in FIG. 7b, the slider 21 is situated between the tongues 20′ and the ring 33.

The elongate hole shown in FIGS. 2 and 4 can also be used in the embodiments with the sleeve 19′.

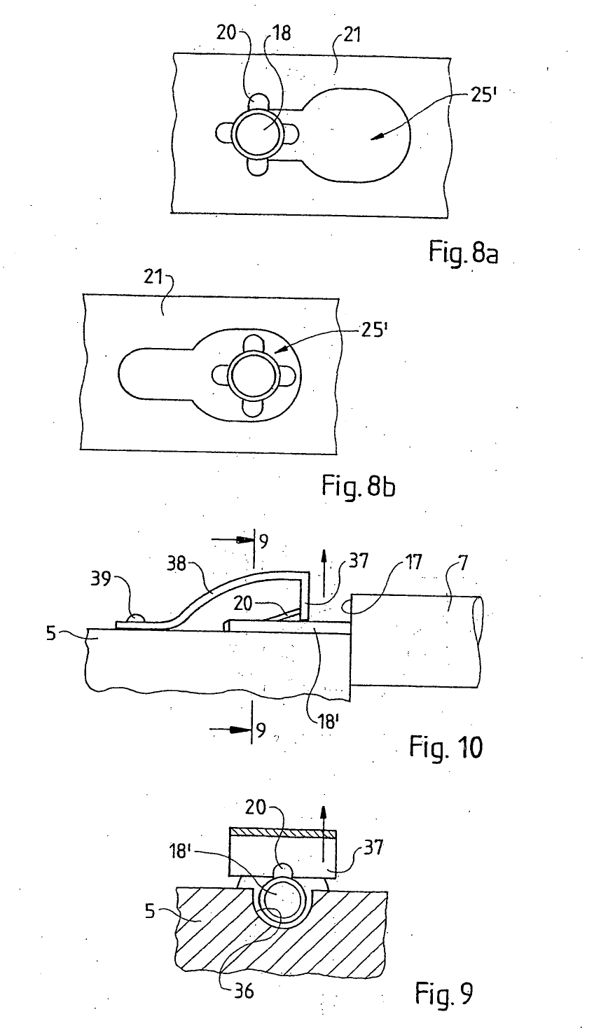

In all the aforementioned embodiments with sleeves 19 or 19′, the elongate hole can also have a shape as is shown in FIGS. 8a and 8b. The elongate hole 25′ in the slider 21 has a small width at its end, which, in the locked position (FIG. 8a) is in engagement with the electrode carrier, so that the tongues find their locking engagement. If it is slid into the position shown in FIG. 8b for the purpose of releasing the slider 21, the tongues 20 are now situated in a substantially broadened region of the elongate hole, through which they can freely pass.

FIGS. 9 and 10 show a further highly simplified embodiment of the electrode carrier fastening. The sliding body 5 does not have, in this case, the receiving bore described in the preceding embodiments as the receiving guide but a receiving groove 36 in one of its side surfaces. Provided on the electrode carrier 7 on the narrowed end member 18 at a predetermined angular position, there is a single tongue 20, which projects beyond the receiving groove 36. The angled end portion 37 of a leaf spring 38 is provided as the locking element which is connected at 39, for example by a rivet to the sliding body 5.

If the leaf spring 38 is gently raised in the direction of the arrow, for example with a finger, the electrode carrier 7 can be withdrawn.

FIG. 11 shows a sleeve 19″, which can be used in one of the preceding embodiments. Instead of the spring tongues which move obliquely in the distal direction described in the preceding embodiments, annular springs 39 are provided in this case, which are cut out in the peripheral direction and project beyond the periphery of the sleeve 19′ and which may be pressed in resiliently against suitable oblique surfaces.

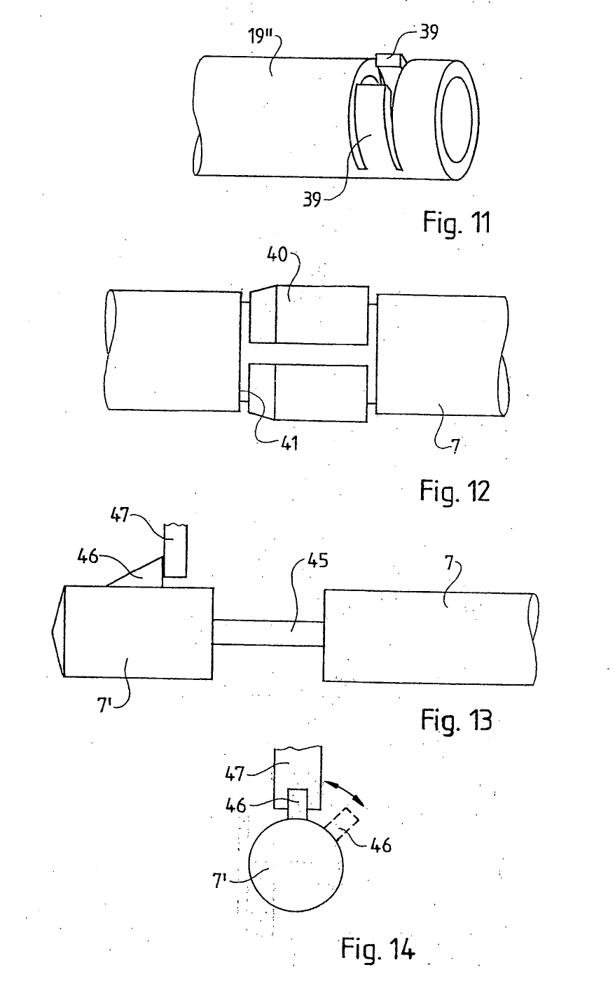

FIG. 12 shows a further embodiment of a spring element, which is mounted in the form of an expanding ring 40 in a peripheral groove 41 in the electrode carrier 7. It is gently bevelled at one (proximal) end in order to be able to move past the edge of the locking element, for example the slider 21 itself, in a manner which applies pressure.

In the embodiments described above, the end stop on the electrode carrier limiting the insertion depth is always constructed in the form of an abutment step 17 on a peripheral thickened portion of the electrode carrier, which abuts against the slider 21. The end stop can also be in a different form. For instance, in the embodiment of FIG. 3, the end member 18 of the electrode carrier 7 can be extended so far that it abuts against the end of the receiving bore 6.

Instead of the abutment step 17 shown in FIG. 6, the abutment step 17′ shown at the proximal end of the sleeve 19′ can also be used as an abutment limiting the insertion depth of the electrode carrier 7 which operates against an abutment, not shown, arranged in the sliding body 5.

In the previously described embodiments, the spring element 20, 20′, 39, 40 is always constructed to yield radially inwards. FIGS. 13 and 14 show a basically different construction. The figures show, in side view in FIG. 13 and in axial view in FIG. 14, the electrode carrier 7 with a proximal end region 7′, which is connected to the distal region 7 of the electrode carrier by means of a torsion element 45. The proximal end region 7′ carries a radially extending projection 46 which, as shown in FIG. 13, catches lockingly behind a locking element 47.

As may be seen in FIG. 14, both the locking element 14 and also the projection 46 have a small width in the peripheral direction. By rotating the region 7′ with respect to the region 7 of the electrode carrier, as is shown in FIG. 14 in the solid and chain line positions of the projection 46, the projection 46 can be moved out of engagement with the locking element 47.

In this manner, locking engagement is thus produced not, as illustrated in the other embodiments, with a radially resilient spring element but with a torsionally resilient spring element. The locking element 47 can have an oblique surface in the distal direction, against which the projection 46 impinges, when the electrode carrier is inserted, in order to be moved laterally by the oblique surface until it can snap back behind the proximal surface of the locking element 47. In order to release the locking engagement, the locking element 47 can be moved out of engagement, for example by movement radially with respect to the axis of the electrode carrier 7 or by lateral movement. For this purpose, it can be provided on a construction similar to the slider 21 (FIG. 2) or provided in some other way fixedly or movably on the sliding body 5 of the resectoscope shown in FIG. 1.

In the previously described embodiments, the locking engagement of the spring element 20, 20′, 39, 40, 45, 46 against the locking edge of the sliding body 5, for example on the slider 21, is always released by moving the edge out of engagement (movement of the slider 21).

A modification is shown in FIG. 6. The slider 21 could be constructed in this case as a fixed locking abutment. A sliding sleeve 48 is provided, which is shown in FIG. 6 out of engagement and which, when moved in the distal direction (to the right in FIG. 6), slides over the sleeve 19′ and compresses the tongues 20′ so that they pass through the hole 25, as shown in FIG. 7a. The sliding sleeve 48 can be moved in the distal direction in a suitable manner, for example with a construction in the manner of the slider 21 or by means of a suitable lifting device.

This constructional principle may also be used in the other embodiments with appropriate modification. This principle is even possible in the embodiment of FIGS. 13 and 14. A body moved in the distal direction, which, with an oblique edge, moves the projection 46 shown in FIG. 14 out of the rotary position shown in solid lines into the rotary position shown in chain lines, can effect unlocking, although the locking element 47 is not moved.

In the embodiment of FIGS. 13 and 14, instead of a separately provided torsion element 45, the electrode carrier 7 can be provided as a torsion element of continuous construction.

1. An electrode (9) for urological resectoscopes including an elongate electrode carrier (7, 18, 19), which is constructed proximately for electrical contact (12) and for mechanical fastening in a receiving guide (6, 36) in a sliding body (5) by a locking connection with a spring element (20, 20′, 39, 40, 45), wherein the spring element (20, 20′, 39, 40, 45) is arranged on the electrode body (7, 18, 19).

2. The electrode as claimed in claim 1, wherein the spring element (20, 20′, 39, 40) is constructed to move under its resilience radially outwardly.

3. The electrode as claimed in claim 2, wherein the spring element has one or more peripherally spaced spring tongues (20, 20′), whose proximal end is connected to the electrode carrier (19) and extend from there distally and obliquely outwardly.

4. The electrode as claimed in claim 3, wherein the spring tongues (20, 20′) are constructed in the form of partial cutouts in a sleeve (19, 19′) connected to the electrode carrier (18).

5. The electrode as claimed in claim 1, wherein the electrode carrier (7) has an end stop (17, 17′) limiting an insertion depth into the receiving guide (6).

6. The electrode as claimed in claim 1, wherein the electrode carrier has a peripherally thickened portion (7, 33) at a distal spacing from the spring element (20, 20′).

7. The electrode as claimed in claim 6, wherein the peripherally thickened portion is constructed in the form of a ring (33), which is slidably mounted on the electrode carrier (7) and is acted on by the proximal end of a longitudinally elastic hose (34), which is mounted on the electrode carrier (7) and whose proximal end is connected to the electrode carrier.

8. The electrode as claimed in claim 1, wherein the spring element (45) carries a radial projection (46), which is rotationally resiliently mounted with respect to the distal region (7) of the electrode carrier.

9. A resectoscope for receiving an electrode, said electrode including an elongate electrode carrier (7, 18, 19), which is constructed proximately for electrical contact (12) and for mechanical fastening in a receiving guide (6, 36) in a sliding body (5) by a locking connection with a spring element (20, 20′, 39, 40, 45), wherein the spring element (20, 20′, 39, 40, 45) is arranged on the electrode body (7, 18, 19), and wherein the receiving guide (6, 36) has, in a fastening region, an edge engaging the electrode carrier (18, 19) proximal of which an empty space is defined adjacent the electrode carrier (18).

10. The resectoscope as claimed in claim 9, wherein the edge is formed on a locking element (21, 38), which is movable out of locking engagement.

11. The resectoscope as claimed in claim 10, wherein the electrode spring element (20, 20′, 39, 40) is constructed to move under its resilience radially outwardly, and wherein the locking element is constructed in the form of a slider (21), which is mounted in the sliding body (5) and is slidable transversely to the electrode carrier (7), and has a hole (25, 25′) which is elongated in a sliding direction and provides the edge at one of its end regions.

12. The resectoscope as claimed in claim 11, wherein the width of the elongate hole (25) corresponds to a diameter of the electrode carrier (19) with the spring tongues (20) moved resiliently inwards, whereby the elongate hole (25), in the locked position of the slider, receives the electrode carrier (18) in its one end region and, at this end region, has a step (26) of increased width on its proximal edge, which tapers obliquely in the direction towards the other end region (27) of the elongate hole (25) to the width thereof.
